(19) United States
(12) Patent Application Publication
(10) Pub. No.: US 2016/0334383 A1
(43) Pub. Date: Nov. 17, 2016

(54) Balling and Weighing Yarn

(71) Applicant: USTER TECHNOLOGIES AG, Uster (CH)

(72) Inventors: Martin Kuster, Eschenbach (CH); Peter Pirani, Grut (CH); Patrik Scheiber, Niederhasli (CH)

(73) Assignee: Uster Technologies, AG, Uster (CH)

(21) Appl. No.: 15/106,451

(22) PCT Filed: Dec. 19, 2014
(86) PCT No.: PCT/CH2014/000180

§ 371 Date: Jun. 20, 2016

(30) Foreign Application Priority Data
Dec. 20, 2013 (CH) 2116/13

(51) Int. Cl. G01N 33/36

(52) U.S. Cl. CPC G01N 33/365

(57) ABSTRACT

The apparatus (1) is used for forming a ball of yarn. It contains a container (2) having an internal volume (21) which is outwardly bounded in the vertical direction partially by an internal wall (31) and in the horizontal direction completely by the internal wall (31). The internal wall (31) has an inlet opening (41) through which the yarn is injectable into the internal volume (21) by means of an air flow. The internal wall (31) is rigid. The internal volume (21) has a downwardly open outlet opening (22) for a yarn ball formed in the internal volume (21). A lower part (33) of the internal wall (31) is inclined downwardly towards the outlet opening (22). By virtue of the rigid construction, the apparatus is simple and cost-effective. Possible problems with moving parts are avoided.

### INTRODUCTION

The present invention lies in the field of testing yarn. It relates to an apparatus for forming a ball of yarn and an apparatus for weighing yarn, according to the preambles of the respective claims.

### DESCRIPTION OF THE PRIOR ART

For determining the yarn count (yarn mass per length or yarn length per mass), a specific length of the yarn to be tested is measured, separated from the rest of the yarn and weighed. The yarn section needs to be brought to the platform of a precision weighing machine for weighing, which advantageously occurs in form of a ball of yarn.

A module for determining the yarn count is described in U.S. Pat. No. 5,025,660 A. In this module, the formation of the ball of yarn occurs in a circular-symmetrical container, which consists of a bell-shaped cap and a plate-shaped weighing platform. The yarn is blown through a tube with an air flow into the cap, said tube opening tangentially in an oblique manner from above into the cap. In the cavity, which is downwardly bounded by the weighing platform and laterally by the cap, an air flow is formed which forms the yarn into a spherical ball of yarn. The injected air escapes through an opening at the upper end of the cap. As soon as the measured yarn length has been reached, the ball remains on the weighing platform. It is lowered onto the weighing machine, whereupon the ball of yarn is weighed. The ball of yarn is then blown away from the weighing platform and the process can be repeated. It is a disadvantage of this apparatus from the prior art that the cap and the weighing platform need to be movable. This leads to higher production costs and a certain susceptibility to errors in operation.

### SUMMARY

It is therefore an object of the present invention to avoid the disadvantages of the apparatus known from U.S. Pat. No. 5,025,660 A. Movable parts are to be avoided in particular.

These and other objects are achieved by the apparatuses in accordance with the invention, as defined in the independent claims. Advantageous embodiments are indicated in the dependent claims.

The invention is based on the idea of providing a rigid or static container for the pneumatic formation of a ball of yarn by an air flow. The container has an internal volume that is externally bounded. The boundary ensures that the yarn remains in the internal volume and is formed into a ball there by the air flow. The internal volume shall additionally comprise a downwardly open outlet opening, through which air can escape on the one hand and the ball of yarn drops from the internal volume onto a weighing machine on the other hand. A bottom part of an internal wall delimiting the internal volume is downwardly inclined towards the outlet opening in order to produce a rolling and/or sliding of the ball of yarn towards the outlet opening when the air flow is cut off.

If reference is made in this specification to a “rigid” container or “rigid” internal wall etc., then this shall mean that the aforementioned items do not comprise any parts that are movable in relation to each other. The rigid items can be produced either integrally or from several fixedly joined parts. In the latter case, they can also be referred to as a “static assembly”.

The terms of “above”, “below”, “horizontal”, “vertical” etc. that are used in this specification relate to the gravitational field of Earth to which the apparatus is subjected in the state ready for operation.

The internal volume shall be delimited partly to the outside in the vertical direction by an internal wall. This means the following: if one moves from specific points in the internal volume in the vertical direction, one meets the internal wall; there are other points in the internal volume on the other hand from which a movement in the vertical direction leads to the outside without any obstruction by the internal wall. Furthermore, the internal wall shall be bounded completely to the outside in the horizontal direction by the internal wall. This means that every movement in the horizontal direction meets the internal wall from any point in the internal volume, with the exception of an inlet opening for the yarn.

The apparatus in accordance with the invention for forming a ball of a yarn contains a container with an internal volume, which is outwardly bounded in the vertical direction partially by an internal wall and in the horizontal direction completely by the internal wall. An inlet opening is formed in the internal wall, through which the yarn is injectable by an air flow into the internal volume. The internal wall is formed in a rigid manner. The internal volume comprises a downwardly open outlet opening for a yarn ball formed in the internal volume. A bottom part of the internal wall is inclined downwardly towards the outlet opening along the entire circumference of the outlet opening.

In one embodiment, the angle of inclination of the bottom part of the internal wall in relation to the horizontal is not greater than −10°. It lies for example between −30° and −10° and is preferably −15°.

The internal volume can be circular-symmetrical about a vertical axis. The term “circular symmetry” is understood in this specification as the rotational symmetry with respect to any arbitrary angle. In one embodiment, the internal wall substantially has the shape of a part of an embedded torus, which is defined by a circle lying in a horizontal plane and whose axis extends vertically, which is open to the inside and whose bottom part is inclined downwardly towards the outlet opening. An embedded torus is the set of all points which have a fixed distance from the aforementioned circle, wherein the fixed distance is smaller than the circle radius.

In one embodiment, the container comprises a central, upwardly open opening which allows air to flow upwardly out of the internal volume. The area of the upwardly open opening is preferably greater than the area of the outlet opening.

In one embodiment, the inlet opening is formed and arranged in such a way that the yarn is injectable through the inlet opening into the internal volume substantially tangentially to the internal wall. The inlet opening can be formed and arranged in such a way that the yarn is injectable through the inlet opening into the internal volume in a direction which is upwardly inclined. The angle of inclination is 5° to 25° for example and is preferably 15° to the horizontal.

During the entrance into the internal volume, the flow velocity of the air is preferably higher than the injection velocity of the yarn, e.g. at least 1.5 times as high.

In one embodiment, the internal wall is made of a thin material which also forms an outer wall of the container. An inlet nozzle, which opens into the inlet opening, can be rigidly connected to the outer wall. The material forming the internal wall and the outer wall is a sheet metal for example, preferably steel sheet or aluminium sheet.

The internal wall can comprise at least one projection protruding into the internal volume. Such a baffle disturbs the formation of a laminar air flow along the internal wall, so that the injected yarn forms a “chaotic” ball and not a ring-shaped roll for example. The at least one projection can be formed in a rigid or flexible way. If several projections are present, they can be distributed regularly or irregularly on the internal wall. They can be identical or differ from each other. Thus, a combination of rigid and flexible projections is possible.

The apparatus in accordance with the invention for weighing a yarn contains the aforementioned apparatus for forming a yarn ball and a weighing machine for weighing a yarn ball formed by the ball formation. The weighing machine is arranged beneath the outlet opening.

In one embodiment, the apparatus additionally contains an air nozzle for blowing the yarn ball away from the weighing machine.

In one embodiment, the apparatus contains additional means for controlling the measuring method and for evaluating the measuring results.

Potential problems with movable parts are avoided due to the rigid configuration of the apparatus in accordance with the invention for forming a ball of yarn. The apparatus is formed in a simpler way and can be produced at lower cost than the one according to the prior art.

### DRAWINGS

A preferred embodiment of the invention will be explained below in closer detail.

FIG. 1 shows an embodiment of the apparatus in accordance with the invention for forming a ball of yarn (A) in a perspective view, (B) in a top view and (C) in a longitudinal sectional view.

FIG. 2 schematically shows cross-sections of different embodiments of the apparatus in accordance with the invention for forming a ball of yarn.

FIG. 3 schematically shows top views of different embodiments of the apparatus in accordance with the invention for forming a ball of yarn.

FIG. 4 shows a further embodiment of the apparatus in accordance with the invention for forming a ball of yarn (A) in a partly exposed perspective view and (B) in a top view.

FIG. 5 shows an apparatus in accordance with the invention for weighing yarn (A) in a perspective view, (B) in a side view and (C) in a longitudinal sectional view.

### DESCRIPTION

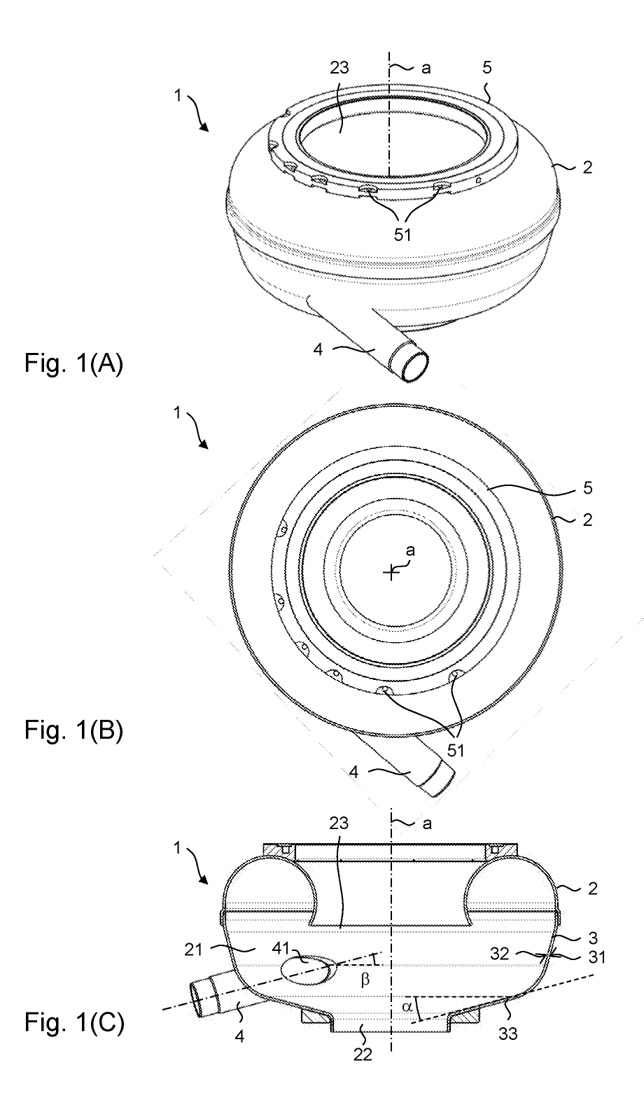

FIG. 1 shows an embodiment of the apparatus 1 in accordance with the invention for forming a ball of yarn (A) in a perspective view, (B) in a top view and (C) in a longitudinal sectional view. The apparatus 1 forms a rigid unit in the sense that it does not comprise any parts that are movable against each other. It can thus be referred to as a static assembly.

The apparatus 1 contains a container 2 with an internal volume 21 which is circular-symmetrical about a vertical axis a. In the embodiment of FIG. 1, the container 2 substantially has the shape of a part of an embedded torus. The container 2 is formed in the present embodiment by a thin wall 3, which is made of a sheet metal for example, preferably steel sheet or aluminium sheet. The wall 3 thus defines an internal wall 31 in the interior of the container 2 and an outer wall 32. The embedded torus is defined by a circle disposed in a horizontal plane, whose central point lies on the axis a. The internal volume 21 is upwardly closed and open to the inside, i.e. towards the axis a. The internal volume 21 comprises a central, downwardly open outlet opening 22 for a yarn ball formed in the internal volume 21. The container 2 similarly comprises a central, upwardly open opening 23. The outer diameter of the container 2 is approximately 270 mm for example, its inner diameter approximately 260 mm, its height approximately 110 mm, and the diameter of the outlet opening 22 is approximately 90 mm and the diameter of the upper opening 23 is approximately 125 mm.

A bottom part 33 of the internal wall 31 is downwardly inclined towards the outlet opening 22 along the entire circumference of the outlet opening 22. The inclination should be at least large enough that a yarn ball is unable to rest on the inclined part 33, but inevitably rolls and/or glides by force of gravity downwardly in an inclined manner and towards the outlet opening 22, and finally leaves the internal volume 21 through the outlet opening 22. Tests have shown that an angle of inclination α of −10° to the horizontal should not be exceeded, i.e. the following should apply: 10°. Exemplary angles of inclination α are −30° to −10° and preferably approximately −15° to the horizontal. The angle of inclination α can change along the circumference of the outlet opening 22.

An inlet opening 41 is disposed in the wall 3 of the container 2. An inlet nozzle 4, which opens into the inlet opening 41, is rigidly connected to the outer wall 32. The inner diameter of the inlet nozzle 4 is 20 to 30 mm for example and preferably approximately 25 mm. The inlet nozzle 4 can also be made of steel or aluminium. A feed hose (not shown) can be connected to the inlet nozzle 4, as known from U.S. Pat. No. 5,025,660 A. The yarn is injected with an air flow into the internal volume 21 through the inlet nozzle 4 and the inlet opening 41. The inlet nozzle 4 and the inlet opening 41 are formed and arranged in such a way that the yarn can be injected into the internal volume 21 substantially tangentially to the internal wall 31. This means that the direction on the internal wall 31 predetermined by the inlet nozzle 4 and the inlet opening 41 is continued without any abrupt changes and without obstructions. In the embodiment shown here, the direction predetermined by the inlet nozzle 4 and the inlet opening 41 is upwardly inclined, namely by an angle β of 5° to 25° and preferably approximately 15° to the horizontal.

In operation, yarn is injected into the interior of the torus part with an air flow through the inlet nozzle 4 and the inlet opening 41. The injection velocity of the yarn can be between 0.5 to 15 m/s for example, the volumetric flow rate of the air 1 to 20 l/s for example, the flow velocity of the air 2 to 40 m/s in the inlet opening 41. An air flow is formed in the internal volume 21, which forms the yarn into a ball in interaction with the internal wall 31. Due to the upwardly facing inclination of the inlet nozzle 4 and the inlet opening 41, the formation of the ball will occur mainly, but not necessarily exclusively, in the upper part of the container 2. The injected air first escapes from the internal volume 21 towards the axis a and then, in the surrounding region of the axis a, in the upward and/or downward direction, through the upper opening 23 or the outlet opening 22. It is advantageous if the upper opening 23 has a greater area than the outlet opening 22, so that the main portion of the air escapes upwardly from the internal volume 21 and thus exerts an upwardly directed force on the yarn to be formed into a ball.

A ring 5 can be fixed, e.g. glued, to an upper part of the container 2, from which air is blown substantially horizontally in the direction of the upper opening 23. The ejection of said air can occur regularly to the inside for example, towards the axis a, tangentially on the circle delimiting the upper opening 23, or in any other direction. It can occur continuously or intermittently, from one direction or from several directions. This injection of air prevents that the yarn is carried out of the torus part with the air escaping from the upper opening 23. The openings 51 which are visible in FIGS. 1(A) and 1(B) on the outer circumference of the ring 5 are used for supplying air through separate lines (not shown). Said air can alternatively be branched off from the air injected through the inlet nozzle 4.

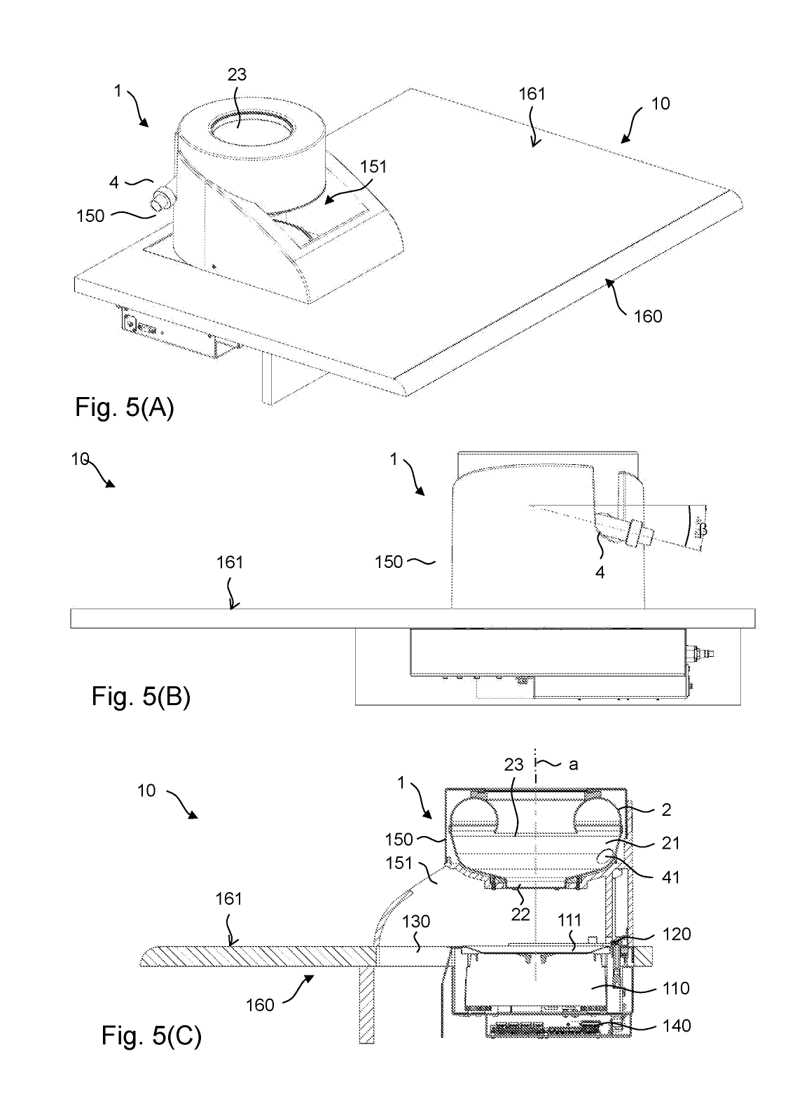

Once the entire yarn section to be formed into a yarn ball is situated in the internal volume 21, the air flow is switched off. The yarn ball then falls downwardly onto the bottom part 33 of the internal wall 31. As a result of the downward inclination of the bottom part 33 of the internal wall 31 towards the outlet opening 22, the ball rolls and/or slides as a result of the force of gravity on the inclined part 33 to an outlet opening 22 and falls through said opening out of the internal volume 21 onto a weighing platform 111 of a weighing machine 110 (see FIG. 5).

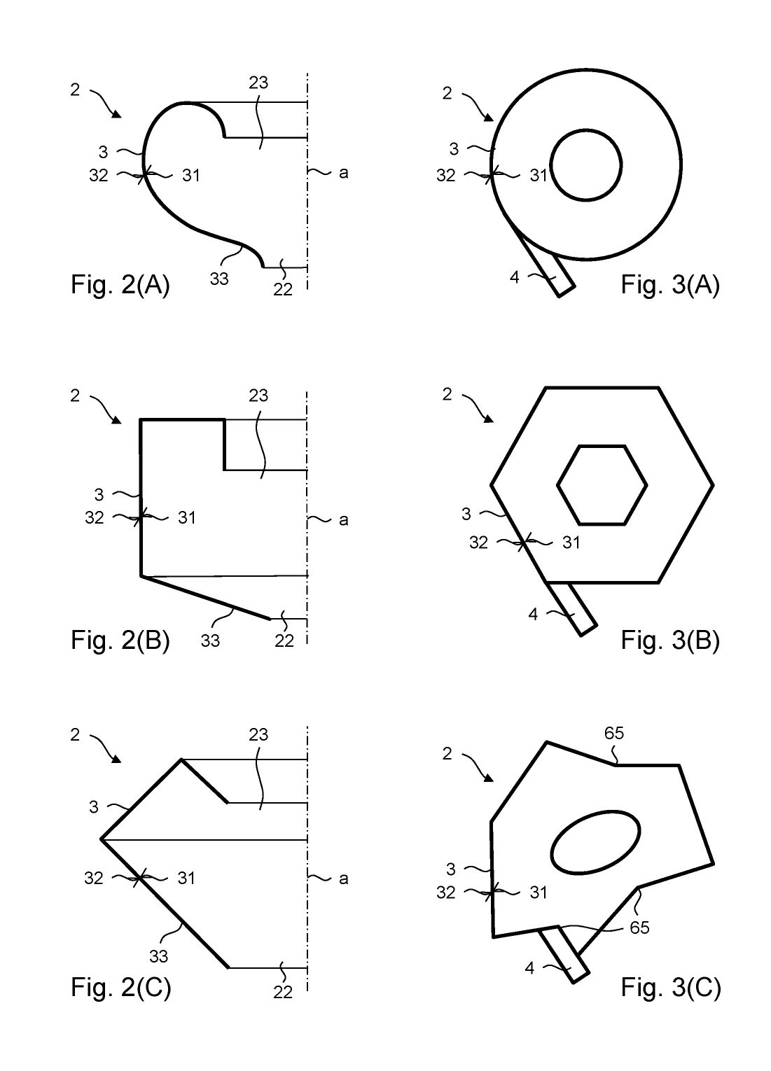

FIG. 2 schematically shows cross-sections of different embodiments of containers 2 of the apparatus 1 in accordance with the invention for forming yarn balls. Due to the circular symmetry of the illustrated embodiments, it is adequate to show only one half of the container 2. The embodiment of FIG. 2(A) approximately corresponds to that of FIG. 1. FIGS. 2(B) and (C) illustrate that shapes of containers 2 other than a part of an embedded torus belong to the invention. Cross-sections with convex or concave corners are certainly possible.

FIG. 3 schematically shows top views of different embodiments of containers 2 of the apparatus 1 in accordance with the invention for forming yarn balls. They are independent of embodiments shown in FIG. 2. The embodiment of FIG. 3(A) approximately corresponds to the one of FIG. 1. FIGS. 3(B) and (C) illustrate that the container 2 need not be circular-symmetric. The container 2 of FIG. 3(B) has a six-fold rotational symmetry, i.e. it forms a regular hexagon in the top view. In the case of the container 2 of FIG. 3(C), no symmetry is present in the top view. The container 2 has the shape of a polygon in the top view with (as seen from the internal volume 21) concave corners and convex corners 65.

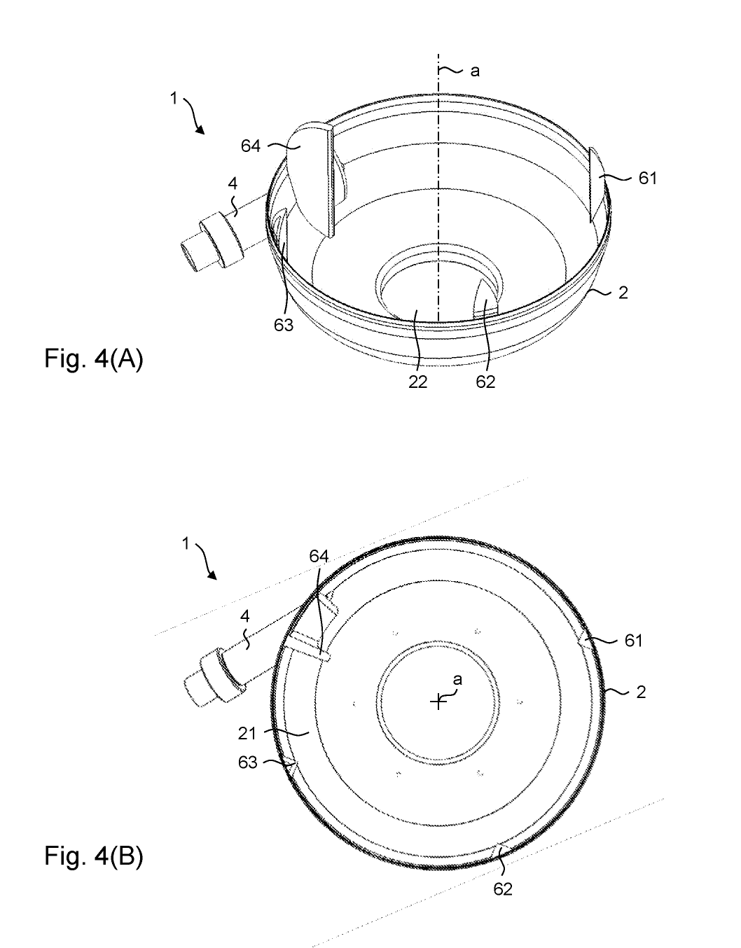

In the embodiment of FIG. 4, the internal wall 31 of the circular-symmetric container 2 comprises several projections 61 to 64 protruding into the internal volume 21. It is their task as baffles to disturb the formation of a laminar air flow along the internal wall 31. Such a laminar air flow is undesirable for the reason that a yarn that is injected therein can form a ring-shaped roll instead of a “chaotic” yarn ball, which roll is unable to leave the inner volume 21 through the outlet opening 22. The projections 61 to 64 can be formed in a rigid or flexible way. In the latter case, they are bent by the air flow in the direction of flow and returned again by their own elasticity. This process is repeated in rapid succession, so that a regularly oscillating or chaotically fluttering reciprocating movement is produced, through which the air flow is disturbed on its part. The projections 61 to 64 can be distributed regularly or irregularly on the internal wall. They can be identical or different among each other. The convex corners 65 in the embodiment of the container 2 according to FIG. 3(C) can also be regarded as projections in accordance with the invention with the aforementioned disturbing function.

FIG. 5 shows an embodiment of the apparatus 10 in accordance with the invention for weighing yarn, which contains the aforementioned apparatus 1 in accordance with the invention for forming a yarn ball, namely (A) in a perspective view, (B) in a side view and

(C) in a longitudinal sectional view. A weighing machine 110 is disposed beneath the apparatus 1 for forming a yarn ball, preferably a precision weighing machine with a weighing platform 111. Neither the weighing machine 110 nor any of its components 111 are part of the apparatus 1 for forming a yarn ball. Accordingly, the apparatus 1 and the weighing machine 110 are vertically spaced from each other, in the embodiment of FIG. 3 by approximately 90 mm.

As described above, the yarn ball drops through the force of gravity onto the weighing platform 111 after the completion of the formation of the yarn ball. It is weighed there by the weighing machine 110. After the weighing process has been completed, the yarn ball can be blown away from the weighing platform 111 by means of at least one air nozzle 120, which can be actuated for this purpose. The yarn ball is preferably blown to a waste opening 130 which leads to a waste container situated beneath (not shown). A respective boundary formed by the encasing 150 is used as a limit stop for the yarn ball.

The apparatus 10 for weighing yarn further contains means 140 for controlling the measuring process and for evaluating the measuring results. The control comprises the activation and deactivation of the air for injecting the yarn into the internal volume 21, the activation and deactivation of the air blown out of the ring 5, the measurement of the length of the yarn section, the cutting of the yarn, the weighing of the yarn ball and/or the blowing away of the yarn ball. The evaluation of the measuring results comprises the calculation of the yarn count from the length and the mass or the weight of the yarn section. In the embodiment of FIG. 3, the means 140 for controlling the measuring process and for evaluating the measuring results is situated beneath the weighing machine 110. The apparatus 1 for forming the yarn ball can be provided with an encasing 150. It is advantageous if the encasing 150 exposes an opening 151, through which an operator can see the weighing platform 111 and can intervene if necessary in order to manually remove any yarn balls that have not been blown away so as to clean the weighing platform 111 etc. The apparatus 10 for weighing yarn can be embedded in a piece of furniture 160, in which or beneath which the aforementioned waste container is located. The piece of furniture 160 preferably comprises a flat horizontal worktop 161, on which a screen (not shown) can be placed among other things.

It is understood that the present invention is not limited to the embodiment as discussed above. With knowledge of the invention, the person skilled in the art will be able to derive further variants which also belong to the subject matter of the present invention.

### LIST OF REFERENCE NUMERALS

1 An apparatus forming a ball of yarn
2 Container
21 Internal volume of the container
22 Outlet opening of the container
23 Upper opening of the container
3 Wall of the container
31 Internal wall of the container
32 Outer wall of the container
33 Bottom part of the internal wall
4 Inlet nozzle
41 Inlet opening in the internal wall
5 Ring on the container
51 Openings on the outer circumference of the ring
61-65 Projections on the internal wall
10 Apparatus for weighing yarn
110 Weighing machine
111 Weighing platform
120 One or several air nozzles
130 Waste opening
140 Control and evaluation means
150 Encasing of the apparatus for forming a ball of yarn
151 Opening in the encasing
160 Furniture
161 Worktop
a Axis of symmetry of the circular-symmetrical internal volume
α Angle of inclination of the bottom part of the internal wall in the direction downwardly to the horizontal
β Angle of inclination of the inlet opening upwardly to the horizontal

1. An apparatus for forming a ball of yarn, comprising:
a container with an internal volume that is outwardly bounded in the vertical direction partially by an internal wall and in the horizontal direction completely by the internal wall
an inlet opening provided in the internal wall through which the yarn is injectable into the internal volume by means of an air flow,
the internal wall formed in a rigid manner,
the internal volume comprising a downwardly open outlet opening for the ball of yarn formed in the internal volume, and
a lower part of the internal wall inclined downwardly towards the outlet opening along the entire circumference of the outlet opening.

2. An apparatus according to claim 1, wherein an angle of inclination of the bottom part of the internal wall to the horizontal is not greater than −10°, and lies between −30° and −10° for example, and is preferably −15°.

3. An apparatus according to claim 1, wherein the internal volume is circular-symmetrical about a vertical axis.

4. An apparatus according to claim 3, wherein the internal wall substantially has the shape of a part of an embedded torus, which is defined by a circle disposed in a horizontal plane and whose axis extends vertically, which is open to the inside, and whose bottom part is inclined downwardly towards the outlet opening.

5. An apparatus according to claim 1, wherein the container comprises a central, upwardly open opening which allows an outflow of air from the internal volume in the upward direction.

6. An apparatus according to claim 5, wherein the area of the upwardly open opening is greater than an area of the outlet opening.

7. An apparatus according to claim 1, wherein the inlet opening is formed and arranged in such a way that the yarn is injectable into the internal volume through the inlet opening substantially tangentially to the internal wall.

8. An apparatus according to claim 7, wherein the inlet opening is formed and arranged in such a way that the yarn is injectable into the internal volume through the inlet opening in an upwardly inclined direction at an angle of from 5° to about 25° to the horizontal.

9. An apparatus according to claim 1, wherein the internal wall is made of a thing material that also forms an outer wall of the container.

10. An apparatus according to claim 9, wherein an inlet nozzle which opens into the inlet opening, is rigidly connected to the outer wall.

11. An apparatus according to claim 9, wherein the material forming the internal wall and the outer wall is a sheet metal.

12. An apparatus according to claim 9, wherein the internal wall comprises at least one projection protruding into the internal volume.

13. An apparatus according to claim 1, further comprising a weighing machine for weighing a ball of yarn formed by the apparatus for forming the ball of yarn the weighting machine disposed beneath the outlet opening.

14. An apparatus according to claim 13, further comprising at least one air nozzle for blowing the ball of yarn away from the weighing machine.

15. An apparatus according to claim 13, further comprising means for controlling the weighing machine and for evaluating an output of the weighing machine.
